(19) United States
(12) Patent Application Publication
(10) Pub. No.: US 2010/0040661 A1
(43) Pub. Date: Feb. 18, 2010

(54) Materials and methods for cell growth

(75) Inventors: John A. Hunt, Liverpool (GB); Judith M. Curran, Liverpool (GB); Duncan Graham, Glasgow (GB); Robert Stokes, Glasgow (GB)

Correspondence Address: FOLEY AND LARDNER LLP;SUITE 500, 3000 K STREET NW, WASHINGTON, DC, 20007, US

(73) Assignee: ULive Enterprises Limited

(21) Appl. No.: 12/458,425

(22) Filed: Jul. 10, 2009

Related U.S. Application Data
(60) Provisional application No. 61/099,182, filed on Sep. 22, 2008.

(30) Foreign Application Priority Data
Jul. 12, 2008 (GB) 0812789.6

(51) Int. Cl. A61F 2/00; C12N 11/14; C12N 11/02; C12N 5/02

(52) U.S. Cl. 424/423; 435/176; 435/177; 435/395

(57) ABSTRACT

Nanopatterned surfaces which provide for improved cell growth including improved stem cell differentiation. The patterned surfaces can comprise an array of fields of biologically active moieties and can be controlled by parameters which include the pitch between the fields and the size of the fields. Nanopatterning can be carried out with use of dip pen nanolithographic printing, microcontact printing, and nanoimprint lithography.

### RELATED APPLICATIONS

This application claims priority benefit to and incorporates by reference in its entirety, including figures, examples, and working examples, claims, and other supporting embodiments (i) UK provisional patent application filing no. 0812789.6 filed on the 12^{th} of Jul. 2008 entitled “Materials and Methods for Cell Growth” filed in the name of ULive Enterprises Limited, and (ii) U.S. provisional application 61/099,182 filed Sep. 22, 2008.

### BACKGROUND

The study and use of biological cells is of considerable interest in a number of clinical and research applications. In many of these applications it is desirable to be able to influence cell behaviour in a selective manner. This may include influencing cell adhesion, cell growth, cell structure (including influencing the development of internal and external cellular structures), and influencing cell differentiation.

A wide variety of means by which cell behaviour may be influenced have been described in the prior art. From amongst these, suitable means may be selected depending on the aim that is to be achieved. One of the most common approaches used is supplementation of cell cultures with growth factors or other biologically active soluble agents. These may be effective in influencing cell behaviour, but are known to suffer from a number of drawbacks. Many cell culture supplements, and in particular growth factors, are expensive, requiring complex purification and/or expression procedures to yield a biologically active agent. Furthermore, many growth factors and supplements are pleiotropic, and may thus give rise to a range of ill-defined effects depending on the concentrations used and the cells to which these factors are provided.

It has recently been shown that functional groups provided on a cell growth substrate may be used to influence cell behaviour. However, this approach also suffers from notable drawbacks. In particular, it has proven difficult to control the biological effects exerted on cells by functional groups used in this way. As a result, cell populations grown in contact with even a single species of biologically active functional groups tend to develop considerable levels of heterogeneity. This is a problem in cases where it is desired to yield substantially pure cell populations, whether for clinical or research purposes.

See Curran et al., Biomaterials, 27 (2006), 4783-4793; Curran et al., Biomaterials 26 (2005), 7057-7067.

### SUMMARY

It is an aim of certain aspects and embodiments of the present invention to obviate or mitigate at least some of the problems associated with the prior art.

The present invention relates to cell growth materials. The invention also relates to methods of influencing the biological activity of a cell; methods of producing cell growth materials; and medical uses of such materials.

In a first aspect, the invention provides a cell growth material comprising a substrate to which are attached a plurality of fields of a biologically active functional group, fields of the biologically active functional group being separated from one another by a region of the substrate that is substantially free from the biologically active functional group, wherein fields and regions define a domain in which the pitch of the fields of biologically active functional groups is substantially constant.

In a second aspect, the invention provides a method of influencing the activities of a biological cell, the method comprising contacting a biological cell with a material in accordance with the first aspect of the invention.

The inventors have found that materials in accordance with the first aspect of the invention are able to influence biological cells that come into contact with the material. Although the ability of biologically active functional groups to influence the activities of biological cells has previously been reported in the literature, this ability has tended to be subject to considerable variability, which may be associated with low levels of reproducibility. This is associated with a failing, in that ceHs grown on prior art materials give rise to populations that are frequently heterogeneous.

In many research or clinical applications it is desirable to utilise cell populations that are substantially homogeneous. Such homogeneous populations may be more thoroughly characterised than heterogeneous populations, and provide more reproducible biological responses. This is important in scientific applications, where elimination of heterogeneity has greater reproducibility of scientific results, and is particularly important in clinical applications, such as cell based therapies.

The inventors have found that materials of the prior art, comprising biologically active functional groups that are capable of influencing the differentiation of biological cells, give rise to cell populations in which the level of heterogeneity may be up to 40% (i.e. the major cell population generated represents as little as 60% of the total cell population). The inventors believe that cell growth materials in accordance with the present invention, and methods utilising such materials, are able to produce cell populations with greatly reduced levels of heterogeneity. The materials and methods of the invention are thus of notable benefit in both clinical and research applications.

Cell-based therapies represent an area that is currently of significant clinical interest. Such therapies typically make use of the ability of biological cells grown ex vivo to augment or replace diseased or damaged tissues when administered to a patient. Cell therapies may make use of substantially differentiated cells (those at or near terminal differentiation) to replace lost or damaged tissues, in which case the cells administered will normally be of the same type as those that it is desired to replace. Alternatively, cell therapies may make use of substantially undifferentiated cells (such as stem or progenitor cells) that are capable of differentiating to produce cells of the required type. In this case, the cells utilised should be capable of differentiation to produce the cell type, or types, needed to achieve the required clinical result.

Stem or progenitor cells represent particularly useful candidates for cell-based therapies. Totipotent or pluripotent stem cells, or multipotent progenitor cells, may all be capable of giving rise to therapeutically useful cell populations. Stem cells may be induced to differentiate and produce therapeutically useful cells in response to external cues provided to the cells either ex vivo or in vivo. Since stem cells of this sort are able to give rise to all, or almost all, cell types within the body they may be expected to provide suitable replacement cells as long as they can be maintained in a substantially undifferentiated state until suitable differentiation cues are provided. The differentiation cues may be provided ex vivo, in order to yield cell populations that can then be administered to a patient, or maybe in vivo in which case naturally occurring or exogenously augmented stem cells may be induced to differentiate by the local milieu, or by artificial agents (such as the cell growth materials of the invention).

In the case of progenitor cells, these will have already undergone a certain amount of differentiation, and this will limit the potential lineages into which they may develop. Accordingly, it is important to be able to produce and select progenitor cells capable of giving rise to replacement cells of the type required. The materials and methods of the invention may be useful in developing substantially homogeneous populations of suitable progenitor cells, and also in ensuring that progenitor cells continue their differentiation along a pathway of interest (for example when materials of the invention are implanted at a site of tissue damage or disease.

In light of the above, it will be recognised that heterogeneities in cell populations, which may cause partial or complete differentiation of certain subpopulations within the whole, are of significant disadvantage since they limit the therapeutic potential of the cells. Indeed, heterogeneities within a population may lead to the generation of cells predisposed to produce a non-useful, or even deleterious, cell type that will not be of any therapeutic benefit.

The inventors have found that a number of these failings of the prior art may be overcome by cell growth materials in accordance with the present invention. These materials utilise biologically active functional groups that are provided in discrete fields separated by regions from which the functional group is absent. To aid the reader's visualisation, in a particular embodiment (as shown in FIG. 1 and FIG. 1a) this may give rise to “dots” of a functional group (the “fields”) on an otherwise uncovered substrate (the uncovered areas providing the “regions” between the fields). That these materials are able to influence the biological activities of cells in a manner that gives rise to more reproducible results, and more homogeneous cell populations, than those found in the prior art is a highly surprising finding. It seems counter-intuitive that the effects of biologically active functional groups may be more evenly exerted on cell populations by an uneven distribution of the functional groups on a surface with which the cells may be brought into contact.

As noted above, the ability of the materials of the invention to give rise to therapeutically useful cell types ex vivo or in vivo is also of notable benefit. Accordingly, the invention also provides the use of a cell growth material in accordance with the first, and subsequent, aspects of the invention as a medicament. Such medicaments may take the form of implantable materials. Implantable cell growth materials in accordance with the invention may be used to provide “conduits” through which therapeutically advantageous cell types may be induced to grow and migrate. Implantable cell growth materials of the invention may also be used to “prime” the replacement or regeneration of damaged tissues, by providing the material on or in which therapeutically useful cell types may be induced to develop. Further details of the medical uses the materials and methods of the invention are described elsewhere in the specification.

The cell growth materials of the invention generally serve to control the binding of specific phenotypicaly defined cells to the materials. Materials that positively influence cell adhesion may be used to promote cell binding, and also to further influence the behaviour of adhered cells, including the differentiation, phenotype or function of such cells. Different properties of cell adhesion, such as the ability to promote adhesion of cell clusters, or of individual cells, may be important in determining the pathways along which biological cells differentiate. For example, clusters of cells may be viewed as indicative of a potential for chondrogenic differentiation, while the development of high adhered, elongated cells (particularly those containing extensive cytoskeletal elements such as stress fibres) may be indicative of a potential for neurogenic or myogenic differentiation. The presence or absence of focal contacts is one of the features that may contribute to cell clustering.

The ability to influence cell differentiation represents a particularly beneficial property of the materials and methods of the invention. It will be appreciated that the manner in which it is desired to influence cell differentiation may vary according to context. In some contexts it may be desired to influence cell differentiation by decreasing the level of differentiation occurring in a population of cells adhered to a material of the invention. This will effectively allow a population of cells to be maintained or expanded without altering their differentiation state. This may be of particular advantage in the case that it is wished to maintain or expand populations of undifferentiated, or substantially undifferentiated, cells (such as totipotent or multipotent stem cells). Generation of large numbers of stem cells is of benefit, since these cells are relatively scarce in adult tissues. The ability to expand stem cell numbers to produce larger populations of these undifferentiated or substantially undifferentiated, cells will be of notable benefit in the therapeutic application of stem cells.

The use of the materials or methods of the invention in the culture of human stem cells may generally represent a preferred embodiment of such aspects of the invention, due to the potential therapeutic utility of such cells, or cell populations derived from such cells. However, it may be preferred that human stem cells to be used in accordance with these preferred embodiments exclude human embryonic stem cells.

In other contexts it may be desired to influence cell differentiation by promoting differentiation to produce a cell lineage of interest. Influencing cell differentiation in this manner is also of benefit in a number of clinical and research applications.

A particular benefit of the ability to influence cell differentiation to produce desired cell types lies in the production of replacement cells that may be used to treat tissues or organs in which a cell type (or cell types) have been depleted through injury or disease. The inventors believe that the materials and methods of the invention may be useful in inducing differentiation along a number of lineages including: osteogenic lineages; chondrogenic lineages; neurogenic lineages; adipogenic lineages; and myogenic lineages. The materials and methods of the invention may be used to induce such lineages from substantially undifferentiated cells, or to further promote the generation of these lineages from cells that are already predisposed to one or more of the lineages in question.

It will be appreciated that the generation of osteogenic lineages will be of interest in the treatment of disease or injuries to bone. Accordingly the materials or methods of the invention capable of giving rise to osteogenic lineages will have particular application in treatment of bone injury or diseases.

The production of chondrogenic lineages will be of particular advantage in the development of cell-based therapies for diseases or injuries of cartilage. Accordingly, materials or methods of the invention that are capable of giving rise to chondrogenic lineages will be of particular benefit in therapeutic applications for diseases or injuries of this type.

Promotion of differentiation to produce neurogenic lineages will be of benefit in the case where it is desired to produce cells to augment or replace nerve cells. Thus materials or methods of the invention capable of giving rise to neurogenic lineages may be of benefit in the treatment of injuries or diseases in which nerve tissue is damaged.

The production of myogenic lineages using the materials or methods of the invention is a benefit when it is desired to produce cells that can give rise to muscle cells (myocytes). Accordingly, materials or methods of the invention capable of giving rise to myogenic lineages will be of particular use in the treatment of injuries or diseases affecting the muscle.

The production of adipogenic lineages using the materials or methods of the invention allows the production of adipose cells that may have a number of therapeutic uses. For example, adipose cells may be used in the reconstruction of structures lost due to trauma or disease. Situations in which this may be of benefit include replacement tissues for use in breast augmentation or remodelling, and the generation of subcutaneous fat layers to be used in association with skin grafting procedures (e.g. after burns or similar injuries). Adipose cells produced using the materials or methods of invention may also be of use in the treatment of congenital defects, such as Poland's syndrome in which there is a lack of natural adipose tissue production.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be further described with reference to the accompanying Figures, in which:

FIG. 1 is a schematic representation of a cell growth material of the invention;

FIG. 2 is a highly magnified schematic representation of a cross-section through a cell growth material of the invention;

FIG. 3 shows lateral force microscopy images of experimental materials of the invention;

FIG. 4 illustrates the results of fluorescent labelling for various cellular components in populations of cells grown on prior art materials comprising biologically active functional groups; and

FIG. 5 illustrates the results of fluorescent labelling for various cellular components in populations of cells grown on cell growth materials in accordance with the present invention.

### DETAILED DESCRIPTION

### Introduction

Various terms that are used in the present disclosure to describe the invention will now be explained further. The definitions and guidance provided below may be expanded on elsewhere in the specification as appropriate, and as the context requires.

All references cited herein are incorporated by reference.

Cells, growth of cells, cell differentiation, and stem cells are generally known in the art. See, for example, Essentials of Stem Cell Biology, (Ed. R. Lanza), 2006; Cell Biology, 2^{nd} Ed., Pollard and Earnshaw, 2008; Gilbert, Developmental Biology, 5^{th} ed., 1997; Cell Lineage Specification and Patterning of the Embryo, (ed. Etkin, Jeon), 2001.

### “Cell Growth Materials”

For the purposes of the present invention, cell growth materials may be taken to comprise any material on or in which a biological cell may be grown. Cell growth materials may be adapted so that they are capable of supporting such growth. Cell growth materials may be used for the growth of cells in vitro, for example in connection with cell or tissue culture; or for the growth of cells in vivo, for example in therapeutic applications in which cell growth materials of the invention are implanted into patients.

Cell growth materials will be capable of supporting cell growth. In the present context cell growth should be taken to encompass both growth of individual cells (in which individual cells grow by a process of differentiation, extension or spreading) and/or expansion of cell numbers (in which a cell population grows by cell replication). Preferred cell growth materials may be capable of promoting both expansion of cell populations, and the growth of individual cells within such populations.

It may generally be preferred that cell growth materials do not incorporate cytotoxic agents or any other agents that will adversely impact the survival of cells, since it will be appreciated that these may impair the growth of cells with which they come into contact.

Cell growth materials in accordance with the invention may, for example, include tissue culture vessels or beads. Cell growth materials may be sterilisable, in order to avoid the risk of contamination of cell cultures, or infection at sites where the materials are implanted. In some circumstances it may be preferred that cell growth materials in accordance with the invention are “wettable”, that is to say adapted to allow their easy acceptance of cell or tissue culture media. Wettability may be influenced by the functional groups utilised in materials or methods of the invention. The wettability of a cell growth material can influence cell adhesion and functionality as disclosed in various prior art publications. It will be appreciated that the influence of wettability on cell adhesion and functionality may be used to increase an inherent property of a material of the invention, or alternatively to “temper” and inherent property by allowing a controlled reduction in the inherent property.

### “Substrates”

Substrates are the component of materials of the invention to which the functional groups are attached. In the case of cell growth materials of the invention, the substrate may provide a surface on which a cell may grow, and/or a matrix or scaffold (for instance a solid or gel-like matrix) through or on which a cell may grow. These embodiments may be referred to as “two dimensional” substrates, or “three dimensional substrates” respectively, for ease of reference. In cases where the substrate provides a surface on which cells may grow, fields of functional groups and complementary regions need only be provided on a surface that will be exposed to cells. In the case of substrates into or through which cells may grow it will be necessary for fields and regions to be defined within the substrate matrix, and optionally on the surface of the matrix.

The inventors have identified a number of substrates that may be used in the materials of the invention, including (but not limited to) those selected from the group consisting of: gold; silica; glass; nitrocellulose; polycaprolactone (PCL); PolyLLactic acid (PLLA); PolyGlycolic acid (PGA); Poly(urethane); hydroxyapatite; tricalcium phosphate; titanium and it's alloys; shape memory alloys and stainless steel.

Suitable substrates may be selected with reference to the intended purpose of the material. Materials of the invention that are intended for use in cell or tissue culture may typically make use of two dimensional substrates conventional in these fields. Such substrates may be relatively inflexible, and may include gold, silica, glass or plastics. Materials of the invention intended for these uses may take the forms of culture vessels (such as flasks, plates, dishes or the like) or beads suitable for use in dispersion culture.

Materials of the invention that are intended for medical use (for example as implantable medicaments, or the like) may utilise the two dimensional substrates, of the sort listed above, or three dimensional substrates. In general, it may be preferred that materials of the invention intended for medical use (and particularly those intended to be implanted) utilise substrates, that are well tolerated within the body, and do not give rise to a chronic inflammatory response. Suitable substrates for use in materials of this sort may be bioresorbable (that is to say the substrate, and/or the material, may be broken down by the body over a period of time), and ultimately replaced with the body's own tissues.

It may generally be preferred to use three dimensional substrates in materials that are to be used in regenerative medicine. Such substrates may serve as scaffolds for the generation of replacement tissues such as bone, cartilage, fat, muscle and neural tissues.

In the case of materials or methods of the invention that are to be used in the generation of replacement bone (for example, materials or methods directed to the production of osteogenic cells) it may be preferred to use substrates having a roughened surface. Roughened surfaces have been shown to be more efficient in bone contacting applications and the expression of osteogenic differentiation markers by functional osteoblast, progenitor and stem cells.

Two dimensional or three dimensional substrates may be selected with reference to a number of parameters that have been shown by the prior art to influence cell attachment and function. These include porosity and pore size; surface chemistry and surface energy; compliance; Young's modulus; pore size; and, in the case of fibrous substrates, orientation of fibres, fibre size and interconnectivity.

### “Fields”

A “field” of functional groups should, for the purposes of the present invention, be taken to encompass any area or volume of a material that that is provided with one or more biologically active functional groups, the area or volume being bounded by at least one region substantially free of said functional group(s). One, or more, further boundaries of a field may be provided by an edge of a material of the invention. A field may comprise only a single biologically active functional group, or may comprise a plurality of functional groups. In the event that a field comprises a plurality of functional groups, the size and shape of the field, and density of functional groups within the field, may be controlled as discussed elsewhere in the specification.

It is surprising that fields comprising only single functional groups are able to influence the adherence, differentiation and other biological properties of cells much larger than the field. It may be expected that fields comprising single functional groups would represent a concentration that was too low to be biologically significant. However, the inventors, while not wishing to be bound by any hypothesis, believe that fields comprising individual biologically active functional groups are sufficient to influence patterns of integrin, which can only in turn exert further influences on the cell.

It may generally be preferred that any given field in accordance with this definition substantially contains only functional groups of a single species. For example, it may be preferred that a field of biologically active functional groups is made up of at least 95% of a single species, preferably at least 96% of a single species, more preferably at least 97% of a single species, even more preferably at least 98% of a single species, or most preferably at least 99% to 100% of a single species.

It may be wished to utilise two or more sets of fields in a material of the invention, each set comprising a different species of functional group or mix of species of functional groups. Thus a material in accordance with the invention may comprise a first set of fields made up of a first species of functional group (or a first mix of species of functional groups) as well as a second set of fields made up of a second species of functional group (or a second mix of species of functional groups). Materials or methods of the invention may make use of third or further sets of fields as required.

It will be appreciated that a field of functional groups may comprise two or more different species of functional group. In this event the combination of species of functional group may be selected based on the properties ascribed to each single species elsewhere in the specification. It may be preferred that different species of biologically active function groups to be combined in the same field be deposited as parts of ink constituents (as described elsewhere) that are able to self-assemble, thus promoting the formation of consistent fields incorporating the different species.

Fields of the invention may have any shape, including (but not limited to) shapes selected from the group consisting of: dots, commas, lines, triangles, squares, crescents and stars. The inventors believe that different shapes of fields may provide benefits in a number of potential applications of the materials or methods of the invention.

For example, the inventors believe that fields having circular patterns may be of benefit in controlling the components of the cytoskeleton, and thereby influencing differentiation of cells exposed to materials provided with such fields. The inventors believe that fields in the shapes of lines or rows will be able to influence single cell morphology, and also impact on population of cells. The use of materials or methods employing fields in the shapes of lines or rows may be useful in the production of neural and myogenic cell lineages. Favoured functional groups which may be employed in the production of such cell lineages are discussed elsewhere in the specification, and it may be particularly preferred to utilise fields of these shapes comprising functional groups shown to contribute to the development of neural or myogenic lineages. Fields in the shape of lines or rows may also influence the production of the extracellular matrix, and thus may be of benefit in the production of cells capable of giving rise to tendon or ligament.

A material or method in accordance with the invention may make use of fields having only a single shape, or may employ fields having a mixture of shapes.

Fields of biologically active functional groups will generally be sized such that at least one of their dimensions (either width or length in the case of fields on substantially flat materials; or width, length or depth in the case of fields in materials in or on which cells may grow) is smaller than the size of a cell to which a material of the invention may be exposed.

It may generally be preferred that at least one dimension of any given field will be less than 100 nm. For example, at least one dimension of any given field may be less than 90 nm, less than 80 nm, less than 70 nm, less than 60 nm or even less than 50 nm. In some embodiments, fields may have at least one dimension that is less than 40 nm, 30 nm, 20 nm or 10 nm. The inventors have found that materials or methods of the invention utilising fields in the form of dots with a diameter of between about 65 nm and 75 nm (most preferably) approximately 70 nm are well suited to achieving the results biological effects described elsewhere in the specification. An average dimension, including an average dot diameter, also can be, for example, less than 100 nm, or less than 90 nm, or less than 80 nm, or less than 70 nm, or less than 60 nm, or less than 50 nm, or less than 40 nm, or less than 30 nm, or less than 20 nm, or less than 10 nm, or, for example 65 nm to 75 nm, or approximately 70 nm.

The inventors believe that the use of fields having a dimension less than 100 nm is advantageous both in terms of the biological effects elicited from cells, and also because this allows the manufacturing time for materials of the invention to be reduced. Reduced manufacturing time is beneficial not only because it allows greater numbers of materials to be produced within a given time, but also because it reduces incidences of uncontrolled diffusion that may otherwise lead to deposition of biologically active functional groups in the areas between fields.

In the case of fields in the shape of lines or rows it will be appreciated that these may incorporate at least one dimension that is considerably longer than 100 nm, and that may be longer than the cells to be exposed to the materials.

“Region of the Substrate that is Substantially Free of a Functional Group”

For the present purposes, a “region of the substrate that is substantially free of a functional group” (also referred to as “a region”, for purposes of brevity) may be any region that is substantially free of a functional group that makes up one or more of the fields present in a material of the invention. In a preferred embodiment, such a region may be substantially free from any functional group, or biologically active functional group. Alternatively, such a region may contain one or more functional groups, so long as these are not functional groups found in a field of the material (i.e. so long as the region is substantially free of any functional groups that make up an individual field as defined above).

A region may be considered substantially free of a given functional group if it is at least 95% free of the functional group, preferably at least 96% free of the functional group, more preferably at least 97% free of the functional group, even more preferably at least 98% free of the functional group; and most preferably at least 99% and up to 100% free of the functional group.

Suitable dimensions of regions in accordance with this definition that may be used in the materials or methods of the invention may be selected with reference to a “pitch” that it is desired to establish between functional group fields. Methods by which pitches between fields may be calculated, as well as preferred pitches for use in the materials or methods of the invention are described further below.

### “Biologically Active Functional Groups”

A “functional group”, for the purposes of the present disclosure, may be considered to comprise any atom or group of atoms which bestows a reproducible chemical functionality upon a compound or molecule incorporating said functional group.

Biologically active functional groups in accordance with the invention are functional groups able to influence the activities of biological cells with which they are brought into contact. Biologically active functional groups may be capable of influencing the adhesion of biological cells, by either increasing or decreasing the level of cell adhesion. Biologically active functional groups may be capable of influencing the differentiation of biological cells, by either promoting or decreasing the level of differentiation occurring. Without wishing to be bound by any hypothesis, the inventors believe that the ability of biologically active functional groups, found in the materials of the invention, to influence cell differentiation may occur as a result of the ability of these functional groups to influence factors such as cell adhesion; focal adhesion formation; cell distribution; and the arrangement, number and distribution of cytoskeletal features.

The ability of a material of the invention, or a biologically active functional group, to promote or decrease an activity (such as differentiation of biological cells) may be assessed with reference to the extent to which the activity in question occurs in control cell populations (for example populations grown in the absence of the functional group in question, and particularly in the absence of a material of the invention comprising the functional group in question).

Examples of biologically active functional groups that the inventors have identified as being suitable for use in the materials or methods of the invention include those selected from the group consisting of: methyl groups; isopropyl groups; cyclohexyl groups; aryl groups; allyl groups; alkynyl groups; hydroxyl (alcohol) groups; ether groups; morpolino groups; ethylene glycosylated groups; polyethylene glycosylated groups; simple sugars, such as glucose, ribose, heparose, or mannose; carboxylate groups; sulphate groups; phosphate groups; phenoxide groups; amino groups; dialkylamino groups; alkylamino groups; phosphine groups; and amino acids.

Isolated biologically active functional groups suitable for use in the materials or methods of the invention may be selected from the group consisting of: methyl groups; isopropyl groups; cyclohexyl groups; aryl groups; allyl groups; alkynyl groups; hydroxyl (alcohol) groups; ether groups; morpolino groups; ethylene glycosylated groups; polyethylene glycosylated groups; carboxylate groups; sulphate groups; phosphate groups; phenoxide groups; amino groups; dialkylamino groups; alkylamino groups; and phosphine groups.

It may be preferred to utilise a hydrophobic functional group in the materials or methods of the invention. Examples of suitable biologically active hydrophobic functional groups may be selected from the group consisting of: methyl groups; isopropyl groups; cyclohexyl groups; aryl groups; allyl groups; and alkynyl groups.

In certain embodiments of the invention it may be preferred to utilise a hydrophilic functional group. Examples of suitable biologically active hydrophilic functional groups suitable for use in the materials or methods of the invention may be selected from the group consisting of: hydroxyl (alcohol) groups; ether groups; morpolino groups; ethylene glycosylated groups; polyethylene glycosylated groups; and simple sugars, such as glucose, ribose, heparose, or mannose.

It may be preferred to make use of a negatively charged functional group in the materials or methods of the invention. Examples of biologically active negatively charged functional groups that may be used in the materials or methods of the invention include those selected from the group consisting of: carboxylate groups; sulphate groups; phosphate groups; and phenoxide groups.

Alternatively, or additionally, it may be preferred to make use of a positively charged functional group. Examples of biologically active positively charged functional groups that may be used in the materials or methods of the invention may be selected from the group consisting of: amino groups; dialkylamino groups; alkylamino groups; phosphine groups; and amino acids.

Biologically active functional groups may be deposited on substrates in the materials of the invention using any suitable chemical compound that makes a suitable functional group available to cells that contact the material. Merely by way of example, a carboxyl group may be deposited by exposure of the substrate to mercaptonehexadecanoic acid (MHA), a methyl group may be deposited by exposure of the substrate to hexadecane thiol (HDT), an amino group may be deposited by exposure of the substrate to 11-amino-1-undecanethiol (AUT), and a hydroxyl group may be deposited by exposure of the substrate to 11-mercapto-1-undecanol (MUOH).

### “Isolated Biologically Active Functional Groups”

It may generally be preferred that the biologically active functional groups used in materials or methods of the invention are isolated biologically active functional groups. For the purposes of the present disclosure “isolated biologically active functional groups”are those that are provided in a form where they are “isolated” from other functional groups. This provides a notable advantage in that cells are not subject to “conflicting” stimuli that may otherwise arise when presented with a number of different species of biologically active functional groups. Thus materials or methods of the invention utilising isolated biologically active functional groups are particularly useful in the production of homogeneous cell populations. Further advantages provided by the use of such isolated biologically active functional groups are considered below.

In accordance with this definition, an isolated biologically active functional group may be any biologically active functional group that comprises the only biologically active functional group provided by an atom, group of atoms, molecule or compound. Thus an isolated biologically active functional group may be a biologically active functional group provided on its own (i.e. not as part of a larger molecule or compound). Alternatively, an biologically active functional group that is part of a larger molecule or compound may also be considered an isolated biologically active group if it is the only functional group provided to a cell by the larger molecule or compound (it will be appreciated that this definition may also encompass biologically active functional groups provided as one of a number of functional groups on a molecule or compound, as long as all of the functional groups are of the same species).

For the purposes of the present invention biologically active functional groups that are part of a mixture of biologically active functional groups present on molecules such as amino acids, or macromolecules such as peptides or nucleic acids, should not be considered to be “isolated”. In these cases the mixture of biologically active functional groups provided by the molecule, macromolecule or compound provides a number of potentially conflicting biological stimuli to cells, and this may result in the generation of cell populations containing substantial levels of heterogeneity.

The inventors believe that the use of isolated functional groups in the materials or methods of the invention provide a number of benefits in addition to those described above. One benefit of the use of isolated functional groups is that, since such isolated groups may be smaller than molecules or macromolecules such as amino acids and proteins, so the density of such functional groups within fields, and the pitch between fields, may be more readily and more precisely controlled compared to biologically active functional groups provided on amino acids, peptides or the like. This size difference may provide a further advantage in that functional groups occurring in amino acids or peptides may extend further from a substrate to which they are attached than do isolated functional groups, thus isolated functional groups may better facilitate cell binding than do their non-isolated counterparts.

The use of isolated functional groups also allows an increased range of functional group species be used, since the range of functional groups available is not constrained by those that are naturally found in amino acids, peptides or the like.

Another benefit of the use of isolated functional groups is the increased longevity of materials incorporating these groups, since isolated functional groups may be provided in forms that are less prone to degradation than are functional groups found as parts of larger biological molecules. These may include isolated functional groups provided on their own (i.e. not as part of a larger molecule) or provided as part of a larger molecule that has greater stability than biological molecules or macromolecules.

The use of isolated functional groups in the materials of the invention, as opposed to functional groups found in larger molecules (such as amino acids, peptides or nucleic acid strands), has a further advantage in that the isolated functional groups are less prone to non-specific binding of constituents of cell culture media. Typically, amino acids or peptides that may include functional groups often bind to media constituents such as those found in foetal bovine serum (a commonly used culture supplement). This non-specific binding can significantly reduce the effectiveness of molecules such as amino acids or peptides, since the biologically active functional groups become “masked” from the cells that they are intended to influence. The extent of non-specific binding occurring is unpredictable, and so the extent of masking of functional groups that may occur in these circumstances can also vary widely. This means that it is difficult to accurately control the amount of an amino acid or peptide made available to cells, and this may contribute to heterogeneous responses in the cell populations exposed to the functional groups. The inventors have found that isolated functional groups suffer from less non-specific binding, and as a result are better able to exert their influence on biological cells in a controlled and predictable manner, thus generating less heterogeneous populations.

The use of isolated functional groups, as opposed to functional groups found in naturally occurring molecules or macromolecules, may also be of benefit in that the isolated functional groups may adhere better to the substrate to which they are attached.

Modifications of functional groups, or molecules or compounds bearing functional groups, able to facilitate the attachment of functional groups and substrates of materials of the invention are discussed elsewhere in the specification.

### “Pitch”

One of the parameters contributing to the arrangement of fields of function groups on the materials of the invention is the pitch of such fields, a measurement that describes the distance between fields on a material, or between fields of the same functional group on a material. The inventors have found that the surprising advantages provided by materials of the invention may be achieved using a range of pitches.

In general, pitches to be used in the materials or methods of the invention may smaller than the size of a biological cell. Preferred pitches between fields of biologically active functional groups for use in the materials or methods of the invention may be in the region of 75 nm to 2000 nm. It is preferred that pitches between fields may be between approximately 100 nm and 1500 nm, and most preferably between 140 nm to 1000 nm. The inventors have found that the use of pitches such as 140 nm, 280 nm or 1000 nm in the materials of the invention provide a number of marked benefits over the prior art, as described in more detail elsewhere in the specification. The use of a 280 nm pitch between fields in the cell growth materials or methods of the invention has been found to be particularly useful. This pitch appears to be associated with favourable cell adhesion activity in all biologically active functional groups investigated to date. Without wishing to be bound by any hypothesis, the inventors believe that the use of fields having a pitch in the region of 280 nm (for example, between about 250 nm and 350 nm) may be particularly effective since the “unevenness” of distribution of functional groups achieved gives rise to very homogeneous responses in populations of cells in contact with such materials. In contrast, smaller pitches or larger pitches may yield reduced responses from biological cells (the fields being either too tightly packed together, or too sparse, to elicit a particularly strong response).

Pitch may be calculated as the distance between centre points of adjacent fields having the same composition of biologically active functional groups. In the case of materials comprising two or more sets of fields (where each set of fields has a different composition of biologically active functional groups) the pitches of the different sets of fields may be the same as one another, or may be different from one another.

Pitch may be measured using techniques such as atomic force microscopy (AFM) and X-ray photoelectron spectroscopy (XPS).

### “Domain”

The materials of the invention contain one or more domains within which the pitch of the fields of biologically active functional groups is substantially constant. Each such domain will comprise a number of fields, preferably 4, 5, 10 or more. A domain may extend across substantially the whole of a material of the invention, or a given material may comprise a number of different domains. In the latter case, the pitch within each domain will be substantially constant, while different domains may share the same pitch or have different pitches.

For the purposes of the present disclosure an area, such as a domain, of a cell growth material may be considered to contain fields having a constant pitch if the pitches within the area do not differ from one another by more than 15%. Preferably the pitches within such an area will differ from one another by no more than 10%, still more preferably by no more than 5%, yet more preferably by no more than 4%, even more preferably by no more than 3%, more preferably still by no more than 2%, and most preferably by 1% or less.

### Methods of Making

The preceding paragraphs set out details of a number of the factors that may be considered in the manufacture of materials in accordance with the invention. In a third aspect, the invention provides a method of manufacturing a cell growth material, the method comprising depositing on a substrate a plurality of biologically active functional groups, to produce a plurality of fields of the biologically active functional group, wherein the fields of the biologically active functional group are arranged such that they are separated from one another by a region of the substrate that is substantially free from the biologically active functional group.

Materials of the invention may be produced (for example by methods in accordance with the third aspect above) using any suitable technology that is capable of depositing function groups on a substrate with sufficient accuracy to produce the fields and regions required. Given that the preferred pitches between fields of function groups will typically be in the region of 75 nm to 2000 nm, and that the fields will typically have a minimum dimension in the region of 100 nm or below, it may be preferred to utilise nano-scale technologies for the production of materials of the invention.

A preferred method that may be used in the production of materials of the invention is dip pen nanolithography (DPN). DPN is a scanning probe lithography technique in which the tips of atomic force microscopes are used to transfer molecules (such as biologically active function groups in accordance with the invention) to a substrate. The atomic force microscope tip effectively functions as a “pen” depositing an “ink” (e.g. the chosen functional group) onto a “paper” (in this case the cell growth substrate). A major advantage of this technology is that large numbers of “pen” heads may be incorporated in DPN devices (in 1D or 2D arrays), thereby allowing nano-scale patterning of relatively large areas of a substrate.

In a preferred embodiment the ink may comprise the functional group as part of a larger molecule (an “ink constituent”) that serves to attach the functional group to the substrate, while making the functional group available to biological cells placed in contact with the material of the invention. Inks of this sort may be referred to as “functional group-presenting inks”. Functional group presenting inks may be selected on the basis of their ability to efficiently present the functional group to biological cells, and also their ability to serve as inks allowing accurate, controlled and reproducible deposition of the functional groups on the substrate.

Properties that will contribute to the ability of an ink to be deposited accurately and reproducibly include viscosity, volatility and vapour pressure. These properties are important in both the ability to apply an ink to a DPN pen, and also in the ability of the ink

to be deposited accurately by the pen onto the substrate. The application of a coating of ink constituents on to a DPN pen will normally be achieved using evaporation or similar technique to provide a suitably even coverage. This provides a reservoir of ink (and thus ink constituents comprising biologically active functional groups) that may be deposited onto the substrate during the production of materials of the invention.

Deposition of the ink from the pen onto the substrate occurs via a water meniscus formed at the contact point between the two components. It is important that this deposition occurs in a controlled manner, and that unwanted diffusion from the meniscus is kept to a minimum. This may be achieved by selection of inks and ink constituents having suitable properties. Desirable properties, and features of ink constituents able to confer such properties, are discussed in more detail below.

Preferred inks may be selected to allow deposition at relatively low temperatures (around room temperature of approximately 22° C.) and at relatively low humidity. Low temperature and humidity typically allows a small water meniscus to be produced at the pen-substrate interface, and this facilitates deposition of ink constituents (and hence biologically active functional groups) in small, well-defined and controlled fields. These conditions, and in particular low temperature, also reduce incidences of uncontrolled airborne diffusion that may otherwise occur from pen to substrate.

When DPN is performed using a mercaptonehexadecanoic acid ink constituent under the low temperature and humidity conditions referred to above, the ink diffusion coefficient is found to be 0.041 μm²/s. The inventors believe that inks having low diffusion coefficients in this range are of benefit, since they allow accurate and reproducible deposition of fields of biologically active functional groups, and reduce the tendency for uncontrolled diffusion that may otherwise reduce accuracy. It will be appreciated that these advantages are desirable irrespective of the biologically active functional group to be deposited.

Preferred inks suitable for use in the production of materials of the invention may also be selected on the basis of their ability to be deposited on a substrate during a relatively short “dwell time” (this being the time that a pen must spend in contact with an area of the substrate to allow the required amount of an ink to be deposited). The use of short dwell times is advantageous since it both reduces the opportunity for unwanted diffusion to occur, and allows more rapid manufacture of the materials of the invention. This benefit of short dwell times is applicable both to DPN performed in intermittent contact mode and DPN performed in contact mode.

The interaction of ink constituents with the meniscus formed between the pen and substrate may be increased by introduction of a polyethylene glycol group in the ink constituent. The inventors have found that this facilitates efficient and consistent deposition of such ink constituents from the pen to a substrate (especially for inks that would otherwise exhibit slow transport from the tip. It may also advantageously increase the volatility of ink constituents. This may be beneficial both in the coating of pens for use in DPN, and in deposition of inks (and hence functional groups) onto the substrate in a controlled and reproducible manner.

Volatility of ink constituents for use in manufacture of the materials of the invention may also be influenced by altering the number of carbon-carbon bonds present in the ink constituent (for instance, in a carbon chain by which the functional group is secured to the substrate). In general, increasing the number of carbon-carbon bonds decreases volatility, while decreasing the number of carbon-carbon bonds increases volatility. The inventors have found that ink constituents comprising a carbon chain with between 10 and 20 carbon-carbon bonds are generally well suited to production of materials of the invention using DPN. Ink constituents comprising carbon chains between 16 and 18 carbon-carbon bonds have been found to be highly beneficial for the deposition of alkyl functional groups, while constituents having approximately 12 carbon-carbon bonds are preferred in inks for deposition of amino or hydroxyl functional groups (since these groups may naturally reduce volatility of ink constituents in which they are incorporated).

In cases where each field comprises a plurality of functional groups, preferred inks to be used in the manufacture of materials of the invention may be capable of self-assembly to produce stable monolayers of ink constituents (and hence biologically active functional groups) attached to a substrate. Self-assembly in this manner promotes stability of the biologically active functional groups, and hence of the materials in which they are incorporated. Self-assembly is facilitated by the presence of interactions between chains and groups in ink constituents (for example alkyl chains interact and also the acid groups may interact in acid inks. A mixture between an acid ink and the an ink comprising hydroxyl groups may also interact by H-bonding, though this will only occur if the alkyl chains are of similar lengths). Accordingly, it may be preferred to utilise ink constituents having carbon chains with carbon-carbon bond numbers at the upper ends of the ranges referred to above in circumstances where it is wished to produce materials of the invention comprising highly stable fields.

From the above it will be recognised that when selecting an ink component for use in the manufacture of materials of the invention is a balance to be struck between factors that contribute to the generation of an ink component that is able to self-assemble, and factors that modulate the volatility and viscosity of the inks in a manner that aids their deposition in a well defined and reproducible manner.

It will be appreciated that many of the criteria discussed above, in the context of constituents for use in DPN, may also be of relevance in other methods by which materials of the invention may be manufactured.

Although DPN represents a preferred technique for use in the manufacture of materials of the invention, any nanolithographic process capable of depositing functional groups in the manner required to produce suitable fields and regions may be utilised. Merely by way of example, other suitable techniques that may be employed include nanoimprint lithography, electron-beam direct-write lithography and direct atomic force microscopy (AFM).

### Other Embodiments

While the materials and methods of the invention may be useful in a wide range of clinical, therapeutic or research applications, as a result of their ability to influence cell activities such as differentiation in a manner that is more reproducible and controlled than may be achieved utilising the prior art, the inventors have identified a number of particularly preferred applications for the materials and methods of the invention, and these are discussed in more detail below.

In a particularly preferred embodiment, the cell growth materials and methods of the invention may be capable of allowing the expansion of stem or progenitor cells, and may do so substantially without inducing differentiation (or, in the case of progenitor cells, further differentiation) of the cells. Expansion of stem or progenitor cell members without inducing differentiation is of great benefit, since it allows the production of large numbers of therapeutically useful cells from small initial numbers of cells. However, though beneficial, this aim has so far proved difficult to achieve using methods known in the prior art. Many growth factors or supplements provided to stem cell cultures to maintain cell viability also cause the cells to undergo lineage commitment and differentiation. Those few cell culture conditions that have thus far been identified that allow cell population expansion without causing differentiation tend to make use of very expensive cytokines (such as FGF2) or even “cocktails” of cytokines (thus further increasing the associated expense).

The inventors have found that materials of the invention utilising fields of methyl (—CH₃) functional groups at a pitch of approximately 280 nm are particularly useful in maintaining stem or progenitor cells in an undifferentiated state. Populations of cells maintained in this way may also be expanded to increase cell members. Materials of the invention in accordance with this embodiment may be produced much more cheaply than previously disclosed conditions (since they do not involve the complex expression and refolding or purification associated with growth factors) and also have a much extended “shelf-life” since the substrates and isolated functional groups are more resistant to degradation than are complex biological molecules such as cytokines. So advantageous are the inventors' findings in this respect that they give rise to further aspects of the invention.

In a fourth aspect, the invention provides a cell growth material for the expansion of stem or progenitor cell populations, the material comprising a substrate to which are attached a plurality of fields comprising a methyl functional group, wherein the fields of methyl functional groups are separated from one another by a region of the substrate that is substantially free from methyl functional groups, and wherein the pitch between fields is between approximately 200 nm and 750 nm. The pitch may preferably be approximately 280 nm.

In a fifth aspect of the invention there is provided a method of expanding a stem or progenitor cell population, the method comprising contacting a stem or progenitor cell with a cell growth material according to the fourth aspect of the invention, and culturing the cell until an expanded population is produced.

The inventors believe that materials or methods of the fourth or fifth aspects of the invention may advantageously make use of PCL substrates, since the inventors' findings have indicated that these substrates have a beneficial effect on the expansion of stem or progenitor cell populations.

The inventors have found that cell growth materials of the invention may be used to influence the differentiation of biological cells so as to produce chondrogenic cells. This may be achieved using stem or progenitor cells that retain the potential to give rise to chondrogenic lineages. It will be appreciated that the ability to produce chondrogenic cells is of use in a number of research or therapeutic applications (for instance in the investigation or treatment of injuries or disease involving damage to cartilage), and this ability also gives rise to further aspects of the invention.

In a sixth aspect of the invention there is provided a cell growth material for the production of chondrogenic cells, the material comprising a substrate to which are attached a plurality of fields of biologically active functional groups selected from the group consisting of: carboxyl groups; methyl groups; and hydroxyl groups, wherein the fields of the biologically active functional group are separated from one another by a region of the substrate that is substantially free from the biologically active functional group.

In a preferred embodiment of such materials, each individual field may be substantially or wholly composed from only a single species of the recited functional groups. However, fields containing two or more species of the recited functional groups may also be employed in useful embodiments of these materials.

In embodiments of these materials using field comprised wholly or substantially of carboxyl groups, it may be preferred that the pitch between fields is of approximately 280 nm. In embodiments of these materials using field comprised wholly or substantially of hydroxyl groups, it may be preferred that the pitch between fields lies in a range between approximately 140 nm and approximately 1000 nm.

In a seventh aspect, the invention also provides a method of producing chondrogenic cells, the method comprising contacting a stem or progenitor cell with a cell growth material in accordance with the sixth aspect of the invention. The method may optionally include culturing the cell until a chondrogenic cell is produced.

Materials and methods of the invention may be used to influence the differentiation of biological cells (in particular stem or progenitor cells) to produce osteogenic cells. This ability is also of significant clinical and research utility (for instance in the investigation or treatment of injuries or disease involving damage to bones), and the ability to influence cell differentiation so as to produce osteogenic cells gives rise to further aspects of the invention.

In an eighth aspect of the invention there is provided a cell growth material for the production of osteogenic cells, the material comprising a substrate to which are attached a plurality of fields of biologically active functional groups selected from the group consisting of: amino groups and carboxyl groups, wherein the fields of the biologically active functional group are separated from one another by a region of the substrate that is substantially free from the biologically active functional group.

In a preferred embodiment of materials in accordance with this eighth aspect of the invention, each individual field may be substantially or wholly composed from only a single species of the amino or hydroxyl functional groups. However, fields containing two or more species of the recited functional groups may also be employed in useful embodiments of these materials.

It may be preferred that materials in accordance with this eighth aspect of the invention utilises pitches between fields of biologically active functional groups that are between approximately 140 nm and approximately 1000 nm.

In a ninth aspect, the invention provides a method of producing osteogenic cells, the method comprising contacting a stem or progenitor cell with a cell growth material in accordance with the eighth aspect of the invention. The method may optionally include culturing the cell until a osteogenic cell is produced.

The materials or methods of the invention have also been found to be able to influence the differentiation of biological cells so as to produce neurogenic cells. This may be achieved using stem or progenitor cells that retain the potential to give rise to neurogenic lineages. It will be appreciated that the ability to produce neurogenic cells is of use in a number of research or therapeutic applications (for instance in the investigation or treatment of injuries or disease involving damage to the nerves), and this ability gives rise to further aspects of the invention.

In a tenth aspect of the invention there is provided a cell growth material for the production of neurogenic cells, the material comprising a substrate to which are attached a plurality of fields of biologically active functional groups selected from the group consisting of: amino groups and hydroxyl groups, wherein the fields of the biologically active functional group are separated from one another by a region of the substrate that is substantially free from the biologically active functional group.

Preferred embodiments of materials in accordance with this tenth aspect of the invention may be consistent with the preferred embodiments described in connection with the eighth aspect above.

A further, eleventh, aspect of the invention provides a method of producing neurogenic cells, the method comprising contacting a stem or progenitor cell with a cell growth material in accordance with the tenth aspect of the invention. The method may optionally include culturing the cell until a neurogenic cell is produced.

The inventors have found that the materials and methods of the invention are able to influence the differentiation of biological cells in a manner such that myogenic cells are produced. This effect may be achieved when the materials or methods are used to grow stem or progenitor cells capable of giving rise to myogenic lineages. The ability to produce myogenic cells will be of benefit in a number of research or therapeutic applications, for instance in the investigation or treatment of injuries or diseases in which muscle cells are damaged. The ability of materials and methods of the invention to produce myogenic cells gives rise to further aspects as follows.

In a twelfth aspect of the invention is provided a cell growth material for the production of myogenic cells, the material comprising a substrate to which are attached a plurality of fields of amino groups, wherein the fields of amino groups are separated from one another by region of the substrate that is substantially free from amino groups.

In a preferred embodiment of this twelfth aspect the fields of amino groups are comprised solely or substantially solely of amino groups. However, other embodiments of this aspect of the invention may make use of fields comprising amino groups in combination with other functional groups.

It may be preferred that materials in accordance with this twelfth aspect of the invention utilise pitches between fields of amino groups that are between approximately 140 nm and approximately 1000 nm.

The invention also provides a thirteenth aspect, a method of producing myogenic cells, the method comprising contacting a stem or progenitor cell with a cell growth material in accordance with the twelfth aspect of the invention. The method may optionally include culturing the cell until a myogenic cell is produced.

In a fifteenth aspect of the invention is provided a cell growth material for the production of adipogenic cells, the material comprising a substrate to which are attached a plurality of fields of hydroxyl groups, wherein the fields of hydroxyl groups are separated from one another by region of the substrate that is substantially free from hydroxyl groups.

It may be preferred that materials in accordance with this fifteenth aspect of the invention utilise pitches between fields of hydroxyl groups that are between approximately 140 nm and approximately 1000 nm.

The invention also provides a sixteenth aspect, a method of producing adipogenic cells, the method comprising contacting a stem or progenitor cell with a cell growth material in accordance with the fifteenth aspect of the invention. The method may optionally include culturing the cell until an adipogenic cell is produced.

The ability to produce cells of adipogenic lineages using the materials or methods of the fifteenth or sixteenth aspects of the invention lends itself to a number of therapeutic uses. Adipose cells produced using these materials or methods may be used in the reconstruction of structures lost due to trauma or disease, and or in the treatment of congenital defects in which there is a lack of natural adipose tissue production. Further examples of such uses are considered elsewhere in the specification.

The inventors believe that materials of the invention comprising polyethylene glycol (PEG) groups may also be of benefit in a range of uses. PEG groups may be deposited using a range of ink constituents, such as 6-PEG-acid, 3-PEG-acid, or 6-PEG-OH. The inventors believe that the “end” functional group on each “PEG” (such as hydroxyl or acid) will play a role in influencing cell behaviour. The presence of PEG groups may also contribute to the generation of a more local hydrated region.

The inventors have found that certain biologically active functional groups can, when provided in a material having fields of the functional groups arranged at particular pitches, prevent the adhesion of biological cells to the material. This finding may be put to use in the development of materials that may be of use in preventing cell adhesion, and so cell growth and colonisation. Indeed, so useful is this finding that it gives rise to a sixteenth aspect of the invention, in which there is provided a material for inhibiting adhesion of biological cells, the material comprising a substrate to which are attached a plurality of fields of a biologically active functional group, wherein the fields of the biologically active functional group are separated from one another by a region of the substrate that is substantially free from the biologically active functional group. Materials in accordance with this sixteenth aspect of the invention may be used to “mask” areas where it is wished to prevent the adhesion and growth of cells.

The inventors have found that materials in accordance with this sixteenth aspect of the invention may be produced using fields of carboxyl or methyl groups at a pitch of approximately 200 nm or below, or a pitch of approximately 350 nm or more.

An seventeenth aspect of the invention provides a method of inhibiting the adhesion of a biological cell, the method comprising contacting a biological cell with a material in accordance with the sixteenth aspect of the invention.

Materials in accordance with this sixteenth aspect of the invention, and/or suitable for use in the seventeenth aspect of the invention, may be produced using the techniques described above in connection with the manufacture of cell growth materials (e.g. dip pen nanolithography, nanoimprint lithography or electron-beam direct-write lithography).

Materials in accordance with the sixteenth aspects of the invention may also comprise fields and regions defining domains in which the pitch of the fields of biologically active functional groups is substantially constant, as considered elsewhere above.

### FURTHER DESCRIPTION OF THE DRAWINGS

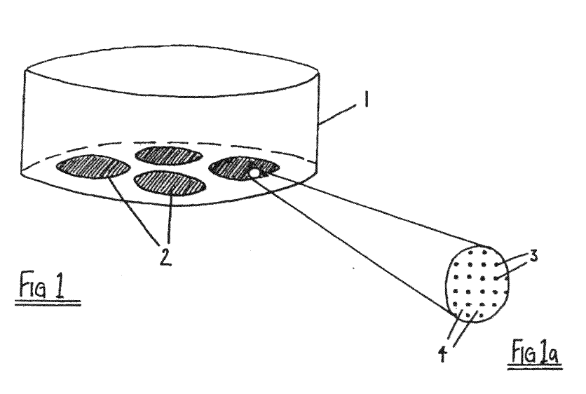

FIG. 1 shows a cell growth material 1 in the form of a cell culture dish. The cell growth material 1 comprises four domains 2. A magnified view of part of one of these domains is shown in FIG. 1a.

Here it can be seen that the domains 2 are comprised of a plurality of fields 3 of a biologically active functional group. These fields 3 are separated by regions 4 of the substrate that are substantially free from the biologically active functional group. The pitch of the fields 3 is substantially constant within the domain 2.

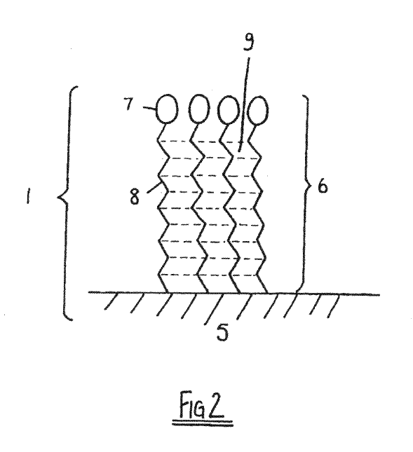

FIG. 2 shows a highly magnified schematic view of a cross-section through a growth material 1 in accordance with a preferred embodiment of the invention. The material comprises a substrate 5. to which are attached a number of ink constituents 6. Each ink constituent 6 comprises a functional group 7 and a carbon chain 8 that serves to attach the functional group 7 to the substrate 5. Carbon chains 8 of adjacent ink constituents 6 interact with one another (interactions represented by dotted lines 9) and thus self-assemble as a monolayer. The monolayer self-assembles such that the functional groups 7 are presented to a biological cell introduced into the area 10 above the cell growth material 1.

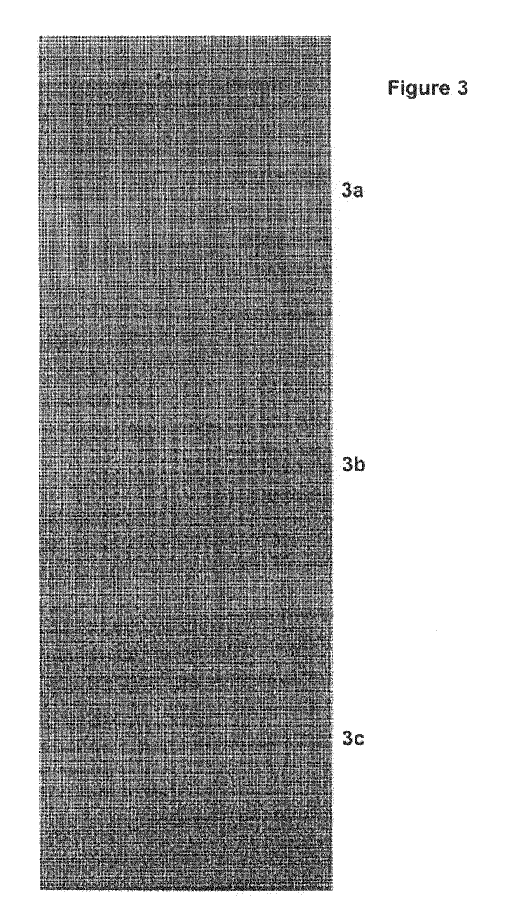

FIG. 3 shows three materials of the invention, comprising fields of carboxyl functional groups, deposited using the ink constituent MHA. Each panel shows a 5 μm by 5 μm region of a material of the invention. The fields have an average diameter of approximately 65 nm to 70 nm, and have pitches of 140 nm, 280 nm and 1000 nm (in

FIGS. 3a, 3b and 3c respectively). The images were obtained using lateral force microscopy (LFM).

Manufacture and uses of the materials of the invention will be further described in the following Experimental Protocols and Results section.

### EXPERIMENTAL PROTOCOLS AND RESULTS

### 1 Study 1

The following study compared the effects on biological cells of materials of the invention with the effects of on biological cells of prior art materials. The results illustrate that cell populations cultured on cell growth materials of the invention exhibited a far greater level of homogeneity (and hence less heterogeneity) than cell populations cultured on prior art materials.

### Protocols

### Preparation of Prior Art Cell Growth Materials

Prior art materials comprising either carboxyl, amino or hydroxyl functional groups were prepared using previously published methods.

Briefly, glass coverslips were dipped into 5% NaOH solution for 1 hour, followed by concentrated HNO3 for 1 hour. The coverslips were then washed in ultra-pure water and 100% ethanol, dried and stored under vacuum prior to attachment of the required biologically active functional groups.

Prior art materials comprising methyl groups were prepared by dipping the cleaned coverslips into dimethyldichlorosilane for 15 seconds, before rinsing with toluene followed by ethanol. The rinsed coverslips were then dried and stored under vacuum before use.

Prior art materials comprising amino groups were prepared by dipping the cleaned coverslips into 0.5% 3-aminopropyltrimethoxysilane isopropyl alcohol solution, then water, and refluxing for 30 minutes. The coverslips were then rinsed with water, followed by ethanol, dried and stored under vacuum before use.

Prior art materials comprising hydroxyl groups were prepared by first coating the coverslips with the vinyl group trimethoxy-vinylsilane using the method described in the preceding paragraph. The vinyl surfaces were then treated with 1M borane-teterdhydrofuran solution for 2 hours in nitrogen. The coverslips were then rinsed in anhydrous tetrahydrofuran before treatment with 0.4% NaOH/30% H2O2 solution for 3 minutes. The coverslips were then rinsed with ultra-pure water followed by ethanol, dried, and stored under vacuum before use.

Preparation of Materials in Accordance with the Invention

Materials of the invention, both experimental cell growth materials and materials capable of inhibiting cell growth using gold substrates and the functional groups described below, were prepared as follows (further details of the reagents used are set out in Appendix 1).

### Experimental Cell Growth Materials

An experimental cell growth material capable of inducing growth of chondrogenic cells was prepared by deposition of fields of carboxyl groups in fields having a pitch of 280 nm. The carboxyl groups were deposited as part of the ink constituent mercaptonehexadecanoic acid (MHA) applied by a dip pen nanolithography (DPN) technique, using a dwell time of 0.1 seconds. Each field comprised a self-assembled area of molecules presenting terminal carboxyl groups in the form of a “dot” with a diameter of approximately 65 nm.

An experimental cell growth material of the invention capable of supporting stem and progenitor cell growth without inducing differentiation was prepared by deposition of methyl function groups in fields at a pitch of 280 nm. The methyl functional groups were deposited as part of the ink constituent hexadecane thiol (HDT) applied via a DPN technique, using a dwell time of 0.1 seconds. Each field comprised an area of self-assembled molecules presenting methyl (alkyl chains) groups in the form of “dot” with a diameter of approximately 75 nm.

Cell growth materials of the invention were prepared comprising fields of amino groups provided at three different pitches. This yielded three materials which had pitches of 1000 nm, 280 nm and 140 nm respectively. The amino groups were deposited as part of the ink constituent 11-amino-1-undecanethiol (AUT) applied using a DPN technique, using a dwell time of 0.2 seconds. Each field comprised an area of self-assembled molecules presenting terminal amino groups in the form of a “dot” with a diameter of approximately 65 nm.

Cell growth materials comprising hydroxyl groups in fields of varying pitches were also produced. Three materials were prepared, having pitches of 1000 nm, 280 nm and 140 nm respectively. The amino groups were deposited as part of the ink constituent 11-mercapto-1-undecanol (MUOH) applied using a DPN technique, using a dwell time of 0.2 seconds. Each field comprised an area of self-assembled molecules presenting terminal hydroxyl groups in the form of a “dot” having a diameter of approximately 65 nm.

Cell growth materials comprising polyethylene glycosylated groups in fields of varying pitches were produced by deposition of 1-(mercaptoundec-11-yl) hexaethyleneglycol (6-PEG-OH) using DPN with a dwell time of 0.2 seconds. Three materials were prepared, respectively having pitches of 1000 nm, 280 nm and 140 nm. Each field comprised a self-assembled area of molecules presenting hydroxyl terminated polyethylene glycol groups in the form of a “dot” with a diameter of approximately 70 nm.

### Experimental Materials for Inhibiting Cell Growth

Materials capable of inhibiting cell growth were prepared by deposition of fields of carboxyl groups (provided as part of the ink constituent MHA applied by DPN) at pitches of either 1000 nm or 140 nm. Each field comprised a self-assembled area of molecules presenting carboxyl groups in the form of a “dot” with a diameter of approximately 65 nm. Further experimental materials of the invention capable of inhibiting cell growth were prepared by deposition of methyl function groups in fields having pitches of 1000 nm or 140 nm. Methyl groups were deposited by the application of the ink constituent HDT using DPN. Each field comprised a self-assembled area of molecules presenting methyl terminating alkyl chains in the form of a “dot” with a diameter of approximately 75 nm.

### Cell Culture

Commercially available bone marrow derived adult human mesenchymal stem cells were characterised using FACS analysis. This characterisation indicated that the stem cells were positive for CD 90, CD173, CD29, CD44, STRO-1 and CD105; and were negative for CD 34, CD24, CD14, CD19 and CD3.

Populations of the characterised stem cells were then seeded onto the experimental materials of the invention described above at a seeding density of 5×10⁴ cells/ml (total). The cells were cultured on the materials in the presence of commercially defined basal medium for 24 hours. The cultured cells were then analysed for cell adhesion, focal contact formation, formation of cytoskeletal components and overall morphology.

### Visualisation of Cell Components

Components of cells cultured on the prior art materials in accordance with the protocols outlined above were visualised by florescence microscopy. F-actin, a major component of the cytoskeleton, was labelled using a green fluorophore, the cell nucleus was visualised using the blue nuclear dye Hoechst, and phenotypically relevant proteins were labelled using a red fluorophore. The selected phenotypically relevant proteins investigated were STRO1 (a stem cell marker) or nucleostemmin (a marker of stem cell proliferation) in cells grown on prior art materials comprising methyl groups, CBFA1 (a marker of osteogenic differentiation) in cells grown on prior art materials comprising amino groups, and collagen II (a marker of chondrogenic differentiation or active chondrocytes) in cells grown on prior art materials comprising hydroxyl groups.

Components of the cells cultured on the materials of the invention in accordance with the protocols outlined above were also visualised by florescence microscopy. The same markers and fluorescent labels were used for cells grown on each of the experimental cell growth materials, as follows: vinculin (a major component of focal adhesions) was labelled using a red fluorophore, F-actin (a major component of the cytoskeleton) was labelled using a green fluorophore, and the cell nucleus was visualised using the blue nuclear dye Hoechst.

### RESULTS

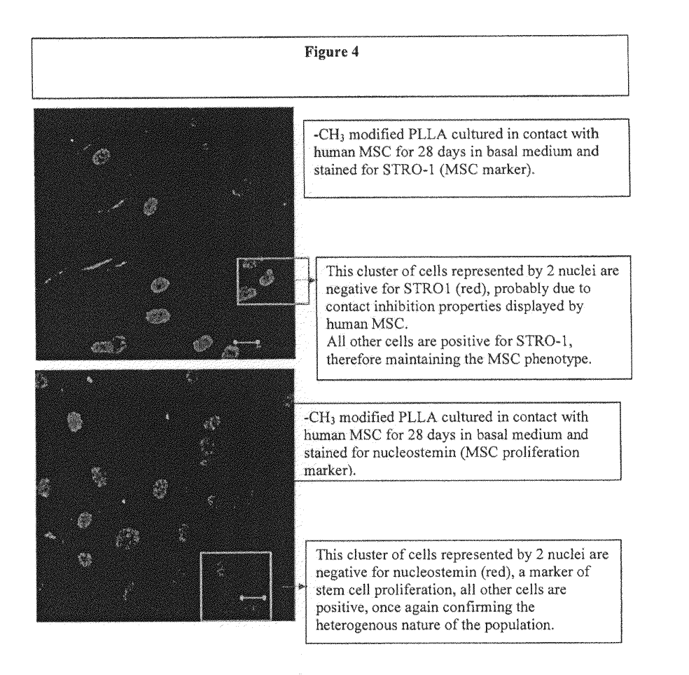

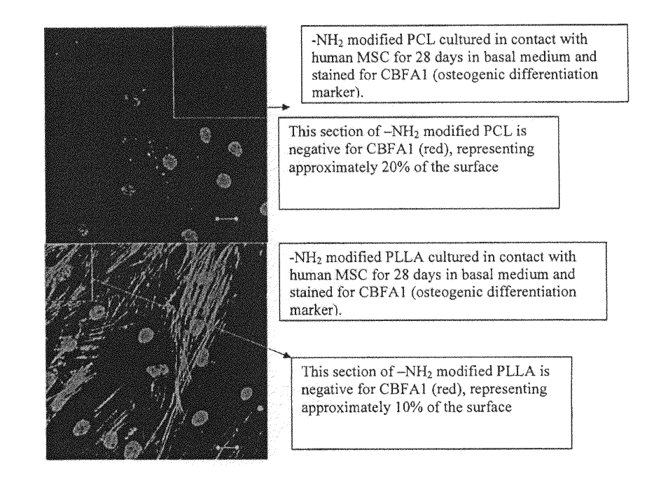

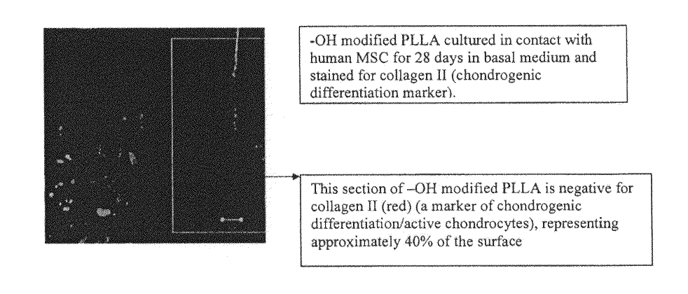

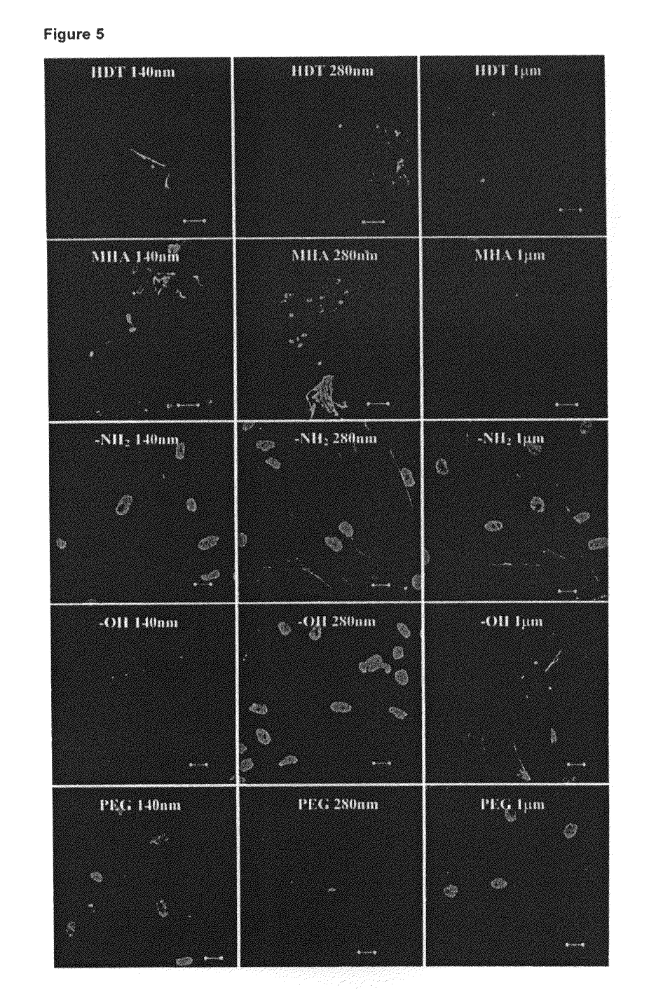

The results of these studies are shown in FIGS. 4 and 5, which set out representative images illustrating co-localisation of various of the cell components listed above, and which are described in further detail below. FIG. 4 illustrates cells grown on prior art materials, whereas FIG. 5 illustrates cells grown on cell growth materials of the invention.

Labelling in cells grown on growth materials of the invention comprising carboxyl groups

Cells adhering on the surface of cell growth materials comprising carboxyl groups took the form of small cell clusters. These clusters comprise well-defined individual cells, with evidence of aligned individual stress fibres within individual cells. Adhered cells have spread out over the surface of the material, and show alignment from cell to cell. Focal contacts are apparent throughout the cell body. These are aligned with the stress fibres and with the ends of the stress fibres, where the outermost protrusion of the cell body contacts the surface of the cell growth material.

Actin had a “ring/halo” formation within individual cells, and there is evidence of actin alignment between cells.

These results indicate that cell growth materials comprising carboxyl groups will promote the chondrogenic differentiation of stem cells, without the need for exogenous biological stimuli.

Labelling in cells grown on prior art materials comprising methyl groups

Populations of cells grown on prior art materials comprising methyl groups were heterogeneous for either the stem cell marker STROL or the marker of stem cell proliferation nucleostemmin. These results indicated that a proportion of stem cells cultured on these materials differentiated (and thereby lost their stem cell status) and/or lost the ability to proliferate.

Labelling in cells grown on cell growth materials of the invention comprising methyl groups

Cells adhered to the cell growth material of the invention comprising methyl groups took the form of cell clusters, individual cells of which exhibited an intact cytoskeletal network and good focal contact formation.

Each cell within cell clusters was nucleated, and focal contacts were present throughout the cluster, at each point where the cluster of cells interacts with the surface of the material of the invention. These focal contacts appear to be responsible for attachment of the cell clusters to the surface of the material. The morphology of individual cells within cell clusters indicates that each cell is well attached to the underlying surface.

A fibrous cytoskeletal network is present throughout the cells, and individual stress fibres are well defined within each individual cell.

Labelling in cells grown on prior art materials comprising amino groups

Cells grown on prior art materials comprising amino groups were found to exhibit inconsistent expression of the osteogenic marker CBFA1. Between 10% and 20% of the total number of cells were negative for this marker. This illustrated that stem cells cultured on prior art materials were incompletely induced to differentiate along an osteogenic lineage, and that the cell populations formed after such culture had a high degree of heterogeneity.

Labelling in cells grown on cell growth materials of the invention comprising amino groups

Cells bound to each of the experimental cell growth materials produced (comprising amino groups provided at pitches of 140 nm, 280 nm or 1000 nm) exhibited massively increased formation of focal contacts and the presence of well organised stress fibres throughout the cell bodies. The cells adhered to these materials were not clustered, and the distribution of the cell nuclei confirms that single cells were well spread and attached to the surfaces of the materials. These materials show the potential to influence orientation of cells, and therefore osteogenic, neurogenic and myogenic differentiation. The pitch of fields may be controlled in order to favour one of these pathways and enhance the kinetics of the overall differentiation reaction.

On all pitches the cells were attached as individual cells in direct contact with the underlying surface. One cell nuclei is evident per cell attached and spread cells have well defined individual stress fibres running throughout the body of the cells. Focal contacts are abundant throughout the cell bodies in close association with individual stress fibres, and at the periphery of the cell bodies once again at the point where the outermost protrusion of a cell interacts with the surface.

Cells on the 280 nm pitch show signs of alignment in a preferential direction, but the overall morphology is still the same as described for all NH₂ modifications.

Labelling in cells grown on prior art materials comprising hydroxyl groups

Stem cells cultured on prior art materials comprising hydroxyl groups gave rise to cellular populations in which the majority of cells were positive for the chondrogenic marker collagen 2, but up to 40% of cells did not exhibit this marker. Accordingly, it can be seen that prior art materials comprising hydroxyl groups exhibit very low efficiency in promoting chondrogenic differentiation, and give rise to highly heterogeneous populations.

Labelling in cells grown on cell growth materials of the invention comprising hydroxyl groups

Cells attached to cell growth materials of the invention comprising hydroxyl groups at all of the pitches investigated. The adhered cells exhibited minimal evidence of focal contact formation, but there was evidence of good cytoskeletal formation throughout the cell bodies. These results indicate that the cell attachment with minimal focal contact formation may be mediated primarily by the presence of hydroxyl groups in the materials of the invention, and that changes of pitch of the fields in turn controls the overall morphology/orientation of the cells. Materials of the invention comprising hydroxyl groups will support cell adhesion and will influence differentiation and induce chondrogenic, adipogenic, osteogenic or neural differentiation in the absence of exogenous biological stimuli. The efficiency of these reactions may be controlled by the pitch of the fields.

Minimal evidence of focal contacts at all pitches but F-actin cytoskeletal components are well formed throughout the cell bodies at all pitches. Cells adhered to materials with a 140 nm or 1000 nm pitch exhibited clear evidence of concentration and “budding” of F-actin components in certain parts of the cell body. Budding is represented by the stress fibres forming a halo like structure around the periphery of the cell and the loss of well defined stress fibres running parallel throughout the body of the cells. The cells are individual, attached and are not forming the clustered morphology previously described on the HDT surface. Cell number is greatest on the materials comprising this functional group at a 280 nm pitch. Cells grown on these surfaces do not display any indication of the “budding” phenomena previously described.

Labelling in cells grown on cell growth materials of the invention comprising PEG

Minimal evidence of focal contact formations on these surfaces, but more than the OH surfaces. Good parallel actin stress fibres are apparent on 280 nm and 1 micron pitch. Stress fibre formation is mainly localised at the periphery of the cell body on the 140 nm pitch, but the stress fibres are still fibres in these regions. Cells are not clustered but are attached on all pitches. Focal contacts are more apparent with individual stress fibres on the 280 nm and 1 micron pitch (2 individual cells attached at certain points).

Materials of the invention for inhibiting cell growth

No cells adhered to or grew upon the experimental materials of the invention for inhibiting cell growth (results not shown).

### 2 Study 2

Commercially available bone marrow derived human mesenchymal stem cells and primary derived stem cells from human blood and dental pulp are cultured on cell growth materials in accordance with the present invention. The cell growth materials comprise various different biologically active functional groups at a range of pitches. The cells are cultured in vitro for time points up to 28 days in basal medium and analysed for expression of a range of markers. The markers investigated are selected from the following group: collagen I, collagen II, collagen X, osteocalcin CBFA1, STRO-1, nucleostemin, beta III tubulin, MAP-2, neurofilament CD90, Synaptophysin and smooth muscle actin. In addition to these markers, staining of cell populations with tinctural stains from the following group: von-Kossa (calcified extra cellular matrix), oil red-o (adipose tissue) and glycosammino-glycan (GAG chondrogenic extra cellular matrix) are investigated.

The markers and tinctural stains selected allow evaluation and analysis of the differentiation capabilities of the cells cultured on the various cell growth materials.

The production of proteins directly related to differentiation capabilities of stem cells is further verified and quantified using western blot, ELISA and real time PCR. The panel of markers used for real time PCR may be selected from those listed above, and also include osteopontin, Sox-9, osteonectin, CHOP (homologous protein), adiponectin and PPAR gamma.

Furthermore, the ability of cell growth materials in accordance with the present invention to induce/maintain a stem cell phenotype over a 28 day in vitro test period under basal conditions will be validated further by culturing stem cells derived from dental pulp and commercially available bone marrow derived human mesenchymal stem cells and transecting with OCT4, SOX 2 (markers associated with embryonic stem cell plasticity but not adult, dental pulp derived do express) and STRO-1 (expressed by adult and embryonic stem cells). The influence of cell growth materials of the invention on expression of these markers will be monitored, and this will provide information regarding the ability of different combinations of functional group and pitch to induce a stem cell phenotype, to maintain selected levels of plasticity, as well as providing information regarding the efficiency and homogenous nature by which any differentiation reactions occur. Transfected cells may be GFP labelled, allowing them to be counterstained with a specified marker of protein production at a given time point.

### APPENDIX 1

DPN materials technical data:
Alkyl functionality:
HDT 1-Hexadecanethiol 674514-500MG [2917-26-2] (More volatile version of ODT)
CH₃(CH₂)₁₅SH
refractive index
n20/D 1.462(lit.)
bp:
184-191° C./7 mmHg(lit.)
mp:
18-20° C.(lit.), 20-24° C.
density
0.84 g/mL at 25° C.(lit.)
ODT 1-Octadecanethiol 01858-100ML [2885-00-9]
CH₃(CH₂)₁₇SH
assay
98%
bp
204-210° C./11 mmHg(lit.)
mp
30-33° C.(lit.)
density
0.847 g/mL at 25° C.(lit.)
OH functionality:
OH 1-Mercapto-1-undecanol 674249-250MG [73768-94-2]
HS(CH₂)₁₁OH
assay
99%
mp
33-37° C.(lit.)
Amino functionality:
NH₃: 11-amino-1-undecanethiol HCl salt 674367 [143339-58-6]
Properties
assay
99%
mp
120-170° C.
Acid functionality:
MHA: 16-mercaptohexadecanoic acid 674435 [69839-68-5]
assay
99%
mp
65-69° C.
PEG functionalities:
PEG-P: (PEG passifier) 1-(mercaptoundec-11-yl) hexaethyleneglycol 675105 [130727-44-5

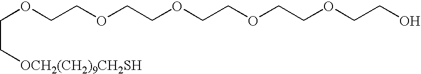

refractive index
n20/D 1.474
density
1.0154 g/mL at 25° C.
PEG-SC: (PEG- dotting for Stem cells) (11-Mercaptoundecyl)tri(ethylene glycol)
673110 [130727-41-2]
assay
95%

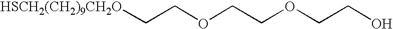

refractive index
n 20/D 1.476
density
0.995 g/mL at 25° C.

In addition several other bespoke molecules were used, e.g. thioctic acid, PEG modified varied chain/PEG lengths.

1. A cell growth material comprising a substrate to which are attached a plurality of fields of a biologically active functional group, fields of the biologically active functional group being separated from one another by a region of the substrate that is substantially free from the biologically active functional group, wherein fields and regions define a domain in which the pitch of the fields of biologically active functional groups is substantially constant.

2. A material according to claim 1, wherein the biologically active functional group is selected from the group consisting of: methyl groups; isopropyl groups; cyclohexyl groups; aryl groups; allyl groups; alkynyl groups; hydroxyl (alcohol) groups; ether groups; morpolino groups; ethylene glycosylated groups; polyethylene glycosylated groups; simple sugars, such as glucose, ribose, heparose, or mannose; carboxylate groups; sulphate groups; phosphate groups; phenoxide groups; amino groups; dialkylamino groups; alkylamino groups; phosphine groups; and amino acids.

3. A material according to claim 1, wherein the pitch between fields of the biologically active functional group is between approximately 75 nm and 2000 nm.

4. A material according to claim 3, wherein the pitch between fields of the biologically active functional group is between approximately 140 nm and 1000 nm.

5. A material according to claim 1, wherein the substrate is selected from the group consisting of: silica; glass; nitrocellulose; polycaprolactone (PCL); PolyLLactic acid (PLLA); PolyGlycolic acid (PGA); Poly(urethane); hydroxyapatite; tricalcium phosphate; titanium; titanium alloys; shape memory alloys and stainless steel.

6. A material for inhibiting adhesion of biological cells, the material comprising a substrate to which are attached a plurality of fields of a biologically active functional group, wherein the fields of the biologically active functional group are separated from one another by a region of the substrate that is substantially free from the biologically active functional group.

7. A material according to any one of claims 1 to 6 for use as a medicament.

8. A cell growth material for the expansion of stem or progenitor cell populations, the material comprising a substrate to which are attached a plurality of fields comprising a methyl functional group, wherein the fields of methyl functional groups are separated from one another by a region of the substrate that is substantially free from methyl functional groups, and wherein the pitch between fields is between approximately 200 nm and 750 nm.

9. A cell growth material according to claim 8, wherein the pitch between fields is approximately 280 nm.

10. A cell growth material for the production of chondrogenie cells, the material comprising a substrate to which are attached a plurality of fields of biologically active functional groups selected from the group consisting of: amino groups, carboxyl groups; methyl groups; and hydroxyl groups, wherein the fields of the biologically active functional group are separated from one another by a region of the substrate that is substantially free from the biologically active functional group.

11. The cell growth material of claim 10, wherein said cells are chondrogenic, osteogenic, neurogenic, myogenic, or adipogenic.

12.-14. (canceled)

15. A material according to any of claims 6-11, wherein fields and regions define a domain in which the pitch of the fields of biologically active functional groups is substantially constant.

16. A cell growth material according to any one of claims 1-11 and 15, wherein the biologically active functional group is an isolated functional group.

17. A method of manufacturing a material according to any one of claims 1-11, 15 and 16, the method comprising depositing on a substrate a plurality of biologically active functional groups, to produce a plurality of fields of the biologically active functional group, wherein the fields of the biologically active functional group are arranged such that they are separated from one another by a region of the substrate that is substantially free from the biologically active functional group.

18. A method according to claim 17, wherein the fields and regions deposited define a domain in which the pitch of the fields of biologically active functional groups is substantially constant.

19. A method according to claim 17 or 18, wherein the biologically active functional groups are deposited by a nanolithography technique.

20. A method according to claim 19, wherein the nanolithography technique is dip pen nanolithography; nanoimprint lithography; direct atomic force microscopy; etching glancing angle deposition; laser ablation; laser deposition; replica molding of x-ray lithography masters; micro contact printing, or etching electron-beam direct-write lithography.

21. A method according to claim 20, wherein the nanolithography technique comprises dip pen nanolithography.

22. A method according to claim 21, wherein the biologically active functional groups are deposited in the form of ink constituents.

23. A method of expanding a stem or progenitor cell population, the method comprising contacting a stem or progenitor cell with a cell growth material according to claim 8, and culturing the cell until an expanded population is produced.

24. A method of producing cells, the method comprising contacting a stem or progenitor cell with a cell growth material in accordance with claim 10, and culturing the cell until a chondrogenic cell is produced.

25. The method of claim 24, wherein said cells are chondrogenic, osteogenic, neurogenic, myogenic, or adipogenic.

26.-28. (canceled)

29. A method comprising dip pen nanolithographically printing a substrate followed by improving growth of at least one cell on the substrate.

30. The method of claim 29, wherein the cell is a stem cell and the improvement is an improved stem cell differentiation or an improved expansion of stem or progenitor cell population.

31. A method comprising:
depositing at least one biologically active compound onto a substrate using a tip to form a plurality of discrete fields of biologically active compound on the substrate,
growing on the substrate comprising the plurality of discrete fields at least one cell until an expanded cell population is produced,
wherein the discrete fields improve a homogeneity or a reproducibility of the cell population compared to growing on the substrate without the discrete fields.

32. The method of claim 31, wherein the substrate without the discrete fields comprises a substantially homogeneous surface comprising the at least one biologically active compound.

33. The method of claim 31, wherein the discrete fields improve a homogeneity of the cell population.

34. The method of claim 31, wherein the growing is an in vivo or an in vitro growing.

35. (canceled)

36. The method of claim 31, wherein the growing does induce differentiation of the cell.

37. The method of claim 31, wherein the growing does not induce differentiation of the cell.

38. The method of claim 31, wherein the cell is a stem cell or a progenitor cell.

39. (canceled)

40. The method of claim 31, wherein the cell is a mesenchymal stem cell.

41. The method of claim 31, wherein the substrate comprising the plurality of discrete fields comprises a pitch between the fields of 75 nm to 2,000 nm.

42. The method of claim 31, wherein the substrate comprising the plurality of discrete fields comprises a pitch of 140 nm to 1,000 nm.

43. The method of claim 31, wherein the substrate comprising the plurality of discrete fields comprises a pitch of 250 nm to 350 nm.

44. The method of claim 31, wherein the substrate comprises the plurality of discrete fields which form a domain, and the domain comprises a substantially constant pitch.

45. The method of claim 31, wherein the discrete fields have at least one dimension less than 100 nm.

46. The method of claim 31, wherein the discrete fields have at least one dimension less than 75 nm.

47. The method of claim 31, wherein the discrete fields are dots with an average diameter of less than 100 nm.

48. The method of claim 31, wherein the discrete fields are dots with an average diameter of 65 nm to 75 nm.

49. The method of claim 31, wherein the discrete fields are dots with an average diameter of at least 65 nm.

50. (canceled)

51. (canceled)

52. The method of claim 31, wherein the substrate is a two dimensional or three dimensional substrate.

53. (canceled)

54. The method of claim 31, wherein the substrate is a silica; glass; nitrocellulose; polycaprolactone (PCL); PolyLLactic acid (PLLA); PolyGlycolic acid (PGA); Poly(urethane); hydroxyapatite; tricalcium phosphate; titanium; titanium alloys; shape memory alloy, or stainless steel substrate.

55. The method of claim 31, wherein the substrate comprises a rough surface.

56. The method of claim 31, wherein the discrete fields comprise at least 95% of a single biologically active compound.

57. The method of claim 31, wherein the discrete fields have dot shapes.

58. The method of claim 31, wherein the discrete fields are separated by regions of the substrate that are substantially free of the biologically active compound.

59. The method of claim 31, wherein the discrete fields are separated by regions of the substrate that are substantially free of any functional group.

60. The method of claim 31, wherein the discrete fields are separated by regions of the substrate that are substantially free of any functional group found in the biologically active compound.

61. The method of claim 31, wherein the biologically active compound comprises at least one hydrophobic group, hydrophilic group, negatively charged group, or positively charged group.

62. The method of claim 31, wherein the biologically active compound comprises at least one functional group which is a methyl group; isopropyl group; cyclohexyl group; aryl group; allyl group; alkynyl group; hydroxyl (alcohol) group; ether group; morpolino group; ethylene glycosylated group; polyethylene glycosylated group; simple sugar, glucose, ribose, heparose, or mannose; carboxylate group; sulphate group; phosphate group; phenoxide group; amino group; dialkylamino group; alkylamino group; phosphine group; or amino acid group.

63. The method of claim 3 1, wherein the biologically active compound comprises at least one isolated biologically active group.

64. The method of claim 31, wherein the tip is a scanning probe tip or an atomic force microscope tip.

65. (canceled)

66. The method of claim 31, wherein the growth produces at least one cell that is a chondrogenic cell, osteogenic cell, neurogenic cell, myogenic cell, or adipogenic cell.

67.-70. (canceled)

71. The method of claim 31, wherein the growth produces a substantially homogeneous population of osteogenic cells, neurogenic cells, myogenic cells, or adipogenic cells.

72.-74. (canceled)

75. The method of claim 31, wherein at least portions of the substrate comprise material to inhibit adhesion of cells.

76. The method of claim 31, wherein the depositing step is carried out with use of nanolithography.

77. The method of claim 31, wherein the depositing step is carried out with use of dip pen nanolithography.

78. The method of claim 31, wherein the homogeneity is improved so that the level of heterogeneity in the expanded cell population is 40% or less.

79. The method of claim 31, wherein the homogeneity or reproducibility is measured in a growing test of at least 24 hours.

80. The method of claim 31, wherein the homogeneity or reproducibility is measured in a growing test of at least 28 days.

81. A method comprising:
providing a plurality of discrete fields of biologically active compound on a substrate,
growing on the substrate comprising the plurality of discrete fields at least one cell until an expanded cell population is produced,
wherein the discrete fields improve a homogeneity or a reproducibility of the cell population compared to growing on the substrate without the discrete fields.

82. The method of claim 81, wherein the providing step is carried out with use of nanolithography.

83. The method of claim 81, wherein the providing step comprises dip pen nanolithography.

84. The method of claim 81, wherein the providing step comprises nanoimprint lithography.

85. The method of claim 81, wherein the providing step comprises microcontact printing.

86. The method of claim 81, wherein the providing step comprises electron beam lithography.

87. The method of claim 81, wherein the providing step comprises use of a scanning probe instrument.

88. The method of claim 81, wherein the providing step comprises use of an atomic force microscope.

89. The method of claim 81, wherein the discreet fields are separated by a substantially constant pitch.

90. The method of claim 81, wherein the providing step is carried out with use of an ink composition comprising the biologically active compound.

91. A method comprising:
depositing at least one biologically active compound onto a substrate using a tip to form a plurality of discrete fields of biologically active compound on the substrate,
growing on the substrate comprising the plurality of discrete fields at least one cell until a differentiated cell population is produced,
wherein the discrete fields improve a homogeneity or a reproducibility of the cell population compared to growing on the substrate without the discrete fields.

92. An article for culturing cells comprising:
a substrate,
a plurality of fields of biologically active molecules on the substrate,
a region devoid of said biologically active molecules between the fields,
wherein cell populations cultured on said article are more homogenous or reproducible than cell populations cultured on a surface of biologically active molecules without fields.

93. An article of claim 92, wherein contact with the fields of biologically active molecules induces differentiation of the cell population.

94. An article of claim 92, wherein contact with the fields of biologically active molecules does not induce differentiation of the cell population.

95. An article of claim 92, wherein contact with the fields induces differentiation into terminally differentiated osteogenic cells, neurogenic cells, adipogenic cells, chondrogenic cell, or myogenic cells.

96. A kit comprising the article of claim 92.

97. The kit of claim 96, wherein the kit comprises instructions for use of the article.
